(19) United States
(12) Patent Application Publication
(10) Pub. No.: US 2002/0086012 A1
(43) Pub. Date: Jul. 4, 2002

(54) COSTIMULATION OF T-CELL PROLIFERATION BY A CHIMERIC BISPECIFIC COSTIMULATORY PROTEIN

(76) Inventors: WINFRIED WELS, RODGAU (DE); BERNHARD GERSTMAYER, KOLN (DE)

Correspondence Address: HELLER EHRMAN WHITE & MCAULIFFE LLP, 4250 EXECUTIVE SQ, 7TH FLOOR, LA JOLLA, CA, 92037, US

(21) Appl. No.: 09/380,415

(22) PCT Filed: Feb. 21, 1998
(86) PCT No.: PCT/EP98/01009

(30) Foreign Application Priority Data
Mar. 4, 1997 (EP) 97103497.0

(51) Int. Cl. A61K 39/395; A61K 39/40; A61K 39/42; A61K 39/00; A61K 39/38; A61K 39/12; C12P 21/08; C07K 16/00

(52) U.S. Cl. 424/134.1; 530/387.3; 530/387.5; 530/387.7; 530/388.22; 530/388.7; 424/135.1; 424/136.1; 424/137.1; 424/138.1; 424/192.1; 424/184.1

(57) ABSTRACT

A soluble bispecific fusion protein consisting of a) a binding domain which recognizes a specific surface molecule on a target cell, covalently linked to b) a domain capable of stimulating T cell proliferation, can be used for a specific costimulation of a T cell directed against said target cell.

This invention pertains to nucleic acids encoding novel chimeric proteins, their corresponding gene products, and their use, whereby the proteins contain two binding domains one of which specifically recognizes a surface molecule on target cells and one of which is derived from the extracellular domain of costimulatory ligands or the counter receptors of such costimulatory ligands naturally expressed on the surface of B lymphocytes, T lymphocytes, or professional antigen presenting cells.

The most effective mechanism for tumor rejection is mediated by tumor-specific T lymphocytes (Greenberg, P. D., 1991, Melief, C. J., 1992). This might favor the progression of tumors which escape immune surveillance by a variety of strategies like the prevention of efficient antigen presentation through the loss of major histocompatibilty complex (MHC) molecules (Doyle, A., et al., 1985, Lassam, N., and Jay, G., 1989) or defects in antigen processing (Restifo, N. P., et al., 1993, Cromme, F. V., et al., 1994). Another mechanism for tumor cells to evade the immune system is the absence of costimulatory molecules (Lundberg, A., et al., 1993). For activation and clonal expansion T cells require costimulatory signals in addition to the primary signal provided by the T-cell receptor (TCR) which interacts with peptide-bearing MHC molecules (Rudd, C. E., et al., 1994). TCR stimulation in the absence of costimulation can result in unresponsiveness and the induction of clonal anergy (Harding, F. A., et al., 1992; Gimmi, C. D., et al., 1993; Tan, P. C., et al., 1993). CD28 is the major costimulatory signal receptor for CD4₊ and CD8₊ T cells . Members of the B7 family of proteins including B7-1 (CD80) and B7-2 (CD86) are its natural ligands on antigen presenting cells (APC) (Gimmi, C. D., et al., 1991; Linsley, P. W., et al., 1991; Galvin, F., et al., 1992: Hathcock. K. S., et al., 1993; Freeman, G. J., et al., Science, 1993; Azuma, M., et al., 1993).

Many attempts have been made to increase tumor immunogenicity. Thereby strategies to provide tumor cells with members of the B7 family of costimulatory molecules have led to promising results. Recently it has been shown that the transmembrane and intracellular domains of B7 molecules are not required for their activity as costimulators of T-cell activation. Expression of the extracellular domain of murine B7-1 or B7-2 on the tumor-cell surface and insertion in the cell membrane via a GPI anchor is sufficient to provide T-cell costimulation in vitro and in vivo (Brunschwig, E. B., et al., 1995). Costimulation of T-cell proliferation in vitro was also achieved upon incorporating in the membrane of tumor cells a recombinant GPI-linked form of human B7-1 which was expressed in CHO cells and purified from cell lysates (McHugh et al., 1995). While this strategy allows to insert a B7 molecule in the cell membrane without the need to transfect the tumor cells with foreign genes, the applicability of such molecules in vivo arc limited by their lack of target cell specificity.

Independent from the availability of professional APCs, T-cell dependent rejection of tumors can be achieved by presenting costimulatory signals directly on the tumor cell surface. Transfection of several murine tumor cells with B7-1 (Chen, L., et al., 1992; Townsend, S. E., and Allison, J. P., 1993; Li, Y., et al., 1994) or B7-2 genes (Hodge, J. W., et al., 1994; Yang, G., et al., 1995) induces T-cell dependent rejection of B7 expressing tumors in mice and protects against subsequent challenge with parental B7-negative tumor cells (Chen et al., 1992; Yang et al., 1995; Baskar, S., ct al., 1993).

Alvarez-Vallina et al. describe, in Eur. J. Immunol. 26 (1996) 2304-2309, an scFv-CD28 fusion gene, its construction, and its functional characterization. The gene product is produced in T cells after gene transfer and is inserted into the cell membrane as a transmembrane protein. It is therefore an immobilized insoluble protein. The fusion protein described by Alvarez-Vallina et al. is a chimeric signal transduction molecule which is produced by the T cell itself.

The invention comprises a novel approach to direct a costimulatory molecule to the surface of target cells. This approach is based on a chimeric fusion protein which consists preferably of the extracellular domain (thus without the transmembrane or intracellular domain) of a costimulatory molecule fused to a single-chain antibody domain (scFv) specific for a tumorspecific antigen, preferably a type I growth factor receptor overexpressed in a high percentage of human adenocarcinomas. Such a molecule is functionally active, soluble and not membrane-located due to the lack of intracellular domain, and binds, for example, to B7 counter-receptors and to ErbB2. The fusion protein localizes specifically to the surface of target cells expressing a tumor-specific antigen, thereby providing a costimulatory signal which results in enhanced proliferation of T cells. The invention shows that effective tumor vaccines for cancer immunotherapy could be created by targeting such chimeric ligands to the surface of tumor cells.

The invention comprises the use of a soluble bispecific fusion protein consisting of

a) a binding domain which recognizes a specific surface molecule on a target cell, covalently linked to

b) a domain capable of costimulation of T cell proliferation, for a specific costimulation of a T cell directed against said target cell.

The invention further comprises a method of manufacturing a therapeutic agent comprising said fusion protein for a specific costimulation of a T cell directed against said target cell of a patient. The therapeutic agent can be administered locally or systemically.

It has surprisingly been found that with the chimeric fusion proteins according to the invention, a very specific cell activation is possible. In contrast to known methods of cell stimulation (e.g., expression of B7 domains on the cell surface), according to the invention, essentially no stimulation of cells in the presence of target cells not carrying the tumor-specific surface antigen is achieved. Therefore, when using methods according to the invention, no background stimulation of cells is found.

The fusion proteins according to the invention consist of two binding domains which do not have any signal transduction function themselves because they contain no intracellular domains, but are in a soluble state when being located outside of cells, and will activate the wild-typical CD28 of a T cell after binding of both the antigen via the scFv domain and of CD28 via the B7 domain, so that said CD28 can generate a signal and transmit it then. Thus, the molecules of the invention provide an antigen-dependent activation of the signal transduction. The fusion proteins according to the invention are typical, because they produce an effect that leads to the stimulation of a specific immune response. The fusion proteins of the invention therefore also are bispecific.

“Target cell” according to the invention preferably means a syngeneic cell, a tumor cell or a cell infected by a pathogen (e.g., virus, bacterium, yeast, fungi).

Therefore, in another embodiment of the invention, a specific activation of, e.g., cytotoxic T cells from a T cell population can also be achieved with the fusion proteins according to the invention, when the fusion protein binds to the specific counter-receptor of the costimulatory domain on the T cell. In such case, it is suitable to carry out a pre-treatment using IL-2.

In another preferred embodiment of the invention, the costimulation can be coupled to ex vivo or in vivo transfection of T cells. Here T cells are transduced with a viral or non-viral gene therapy vector containing a desired gene, the transfected cells being selected, e.g., through a positive or negative selection system. Thereafter, the T cells, which are still at rest, are stimulated through a costimulatory signal, preferably in accordance with the invention, and their proliferation in vitro or in vivo is initiated.

According to the invention a costimulatory molecule is directed to the surface of target cells, which are preferably based on providing the extracellular, CD28-binding domain of human B7-2 with a target-cell specific recognition domain. Consistent alterations of cell surface antigens have been identified in human cancer cells. The erbB2 gene encodes a 185-kDa transmembrane glycoprotein that is a member of the type I family of receptor tyrosine kinases (RTK) which also includes epidermal growth factor (EGF) receptor, ErbB-3 and ErbB-4 (Peles, E., and Yarden, Y., 1993). Overexpression of ErbB2 is frequently observed in human adenocarcinomas arising at numerous sites including breast, ovary, lung, stomach and salivary gland where it correlates with an unfavorable patient prognosis (Hynes, N. E., 1993). Its role in cancer development and its accessible location on the cell surface make ErbB2 a target for directed therapy. From the mRNA of hybridoma cells producing a monoclonal antibody specific for the extracellular domain of human ErbB2 previously a recombinant single chain (scFv) antibody domain consisting of the variable domains of the antibody heavy and light chains connected via a synthetic linker sequence was constructed (Wels, W., et al., 1992). This recombinant binding domain was incorporated in several fusion proteins and has been used to target heterologous effector functions such as enzymes or toxins or gene-transduced cytotoxic T cells (Moritz, D., et al., 1994) specifically to ErbB2 expressing tumor cells (Wels, W., et al., Bio/Technology, 1992; Wels, W., et al., Cancer Res., 1992).

While these strategies are independent of an existing anti-tumor immune response and can be effective in eliminating an established tumor (Wels et al., Cancer Res., 1992), they do not provide long term protection or systemic immunity which could prevent possible tumor recurrence. In contrast, molecules according to the invention might support the generation of a specific T-cell dependent anti-tumor immune response. The invention shows that such a chimeric protein is bifunctional: it localizes specifically to the surface of ErbB2 expressing target cells via the scFv domain and interacts with soluble or cell-surface CTLA-4. Likewise the fusion protein bound to the surface of Jurkat cells which express low levels of CD28 as determined by FACS analysis. In the in vitro experiments with ErbB2 expressing target cells the chimeric proteins according to the invention provided costimulation of PMA-activated syngeneic T cells via the B7 domain.

Cell-surface targeted fusion proteins according to the invention were able to costimulate preactivated T cells. Previously a soluble B7-1-Ig fusion protein at concentrations of 1 to 10 μg/ml showed only modest enhancement of T-cell proliferation in combination with an anti-CD3 antibody, but was more active when immobilized on a plastic surface (Linsley et al., 1991). The most likely explanation for these findings is that CD28 molecules have to be clustered on the surface of the T cell to reach a certain threshold for T-cell activation (Ledbetter, J. A., et al., 1990). Recently it has been demonstrated that a disulphide-linked CTLA-4 homodimer binds to two molecules of monomeric B7-1 or B7-2 with a very fast off-rate (Linsley, P. S., et al., 1995). It is very likely that this is also the case for the binding of CD28 to members of the B7 family, which occurs with an even faster off-rate for B7-2 compared to B7-1 (Linsley, P. S. et al., 1994). B7 fusion proteins anchored on the target cell surface via the antibody domain or membrane-anchored B7 molecules in general simultaneously provide multiple contacts with CD28 molecules which could stabilize the interaction and result in CD28 crosslinking.

The invention shows that the extracellular domain of a costimulatory molecule, preferably human B7-2, targeted to the surface of cells via an antibody domain is able to provide a costimulatory signal for the activation of T cells. Recent work in murine model systems suggests that B7-1 transfected tumor cells might be more effective than those transfected with the B7-2 gene in activating T cells (Gajewski, T. F., et al., 1996; Matulonis, U., et al., 1996). In a recent report where the extracellular domains of murine B7-1 and B7-2 were expressed as GPI-anchored proteins on the surface of the T-lymphoma line EL-4, B7-2 expressing EL-4 transfectants appeared to be at least as potent in enhancing the proliferation of PMA-stimulated primary T cells as B7-1 transfectants (Brunschwig et al., 1995).

A recombinant B7-2₂₂₅ protein (amino acids 1-225) in an E. coli expression system has also been provided which failed to bind to B7 counter-receptors. In contrast, according to the invention, biologically active, soluble lymphocyte receptors and their ligands can be produced in the yeast Pichia pastoris. The B7-2₂₂₅ and B7-2₂₂₅-scFv(FRP5) proteins as well as a truncated human CTLA-4 molecule purified from Pichia pastoris culture supernatants showed specific binding to their respective receptors.

According to the invention, a functionally active B7-2 fusion protein can be targeted to the surface of tumor cells via a specific binding domain. Due to the modular structure of this fusion protein also similar molecules with altered target cell specificity or containing a different immunomodulatory domain could be obtained. Such molecules are therefore useful reagents for cancer immunotherapy.

Preferred binding domains which recognize surface antigens on target cells include growth factor domains or recombinant antibody domains (e.g., single chain Fv domains; disulphide bridged Fv domains) specific for members of the ErbB family of receptor tyrosine kinases such as EGF receptor, variant EGF receptor (EGFRvIII), ErbB2 (HER2, Neu), ErbB3 (HER3), ErbB4 (HER4), which are overexpressed on a variety of tumor cells of epithelial origin. Alternatively, these binding domains can bind to different molecules with enhanced or exclusive expression on the surface of target cells such as tumor cells, or cells infected by pathogens. Such binding domains include binding domains which bind to other growth factor and cytokine receptors, or domains which bind to receptors for peptide ligands such as alpha-MSH expressed on the surface of melanoma cells, or domains which bind to surface molecules which are not receptors for growth factors, cytokines or peptide ligands such as EGP-2, a 38 kDa pancarcinoma antigen recognized by the monoclonal antibody MOC-31, or domains which bind to antigens of pathogens expressed on the surface of infected host cells.

Preferred domains of costimulatory ligands and counter receptors of costimulatory ligands are derived from B7-1 (CD80), B7-2 (CD86), B7-3, CD40, CD11a/18 (LFA-1), CD19, CD22, CD58 (LFA-3), CD59, CD54, CD106 (VCAM), CD72, CTLA-4, CD28, CD40 ligand (CD40L), CD54 (ICAM-1), CD45RO, CD43, CD49d/29, CD5 which are expressed on the surface of B lymphocytes, professional antigen presenting cells, or T lymphocytes.

Recombinant chimeric molecules containing at least two binding domains are derived by isolating nucleic acids encoding binding domains recognizing a surface molecule on target cells, and nucleic acids encoding binding domains derived from costimulatory ligands or their counter receptors, and fusing such nucleic acids in a single open reading frame.

Chimeric molecules according to the invention, containing binding domains which recognize a surface molecule on target cells such as tumor cells, or cells infected by a pathogen, and binding domains derived from costimulatory ligands (e.g., B7-1, B7-2), may act as a vaccine and specifically localize to the surface of a target cell thereby providing the target cell with the costimulatory activity and facilitating a target-cell specific cellular immune response (e.g., by costimulating the activation of T lymphocytes). This could be achieved by systemic treatment of a mammal (patient; farm animal; pet animal) with such a chimeric fusion protein, or by injection of the chimeric fusion protein into a tumor, or by injection of the chimeric fusion protein into tissue infected by a pathogen. Alternatively such chimeric fusion proteins could be used ex vivo for the activation of patient-derived tumor infiltrating lymphocytes (TILs), or lymphokine-activated killer cells (LAK), or other patient-derived lymphocyte preparations in the presence of cells expressing the target antigen, possibly in addition to the presence of activating cytokines such as interleukin 2, interleukin 12, etc, followed by adoptive transfer of such activated lymphocytes into a patient. Alternatively such patient-derived lymphocytes may consist of lymphocytes transduced with chimeric antigen receptors (e.g., nucleic acids encoding chimeric proteins which consist of a binding domain specific of the same antigen on the surface of the target cells recognized by the chimeric costimulatory molecule, or a binding domain recognizing a different antigen on the surface of the target cells, and an intracellular domain derived from molecules such as the zeta-chain or other molecules of the CD3 complex).

The following examples, references, sequence listing and drawings are provided to aid the understanding of the present invention, the true scope of which is set forth in the appended claims. it is understood that modifications can be made in the procedures set forth without departing from the spirit of the invention.

### DESCRIPTION OF THE FIGURES

FIG. 1 Construction and expression in yeast of recombinant B7-2 proteins. (A) Schematic representation of B7-2 genes in the yeast expression plasmids pPIC9-B7-2₂₂₅ (upper panel) encoding amino acids 1 to 225 of human B7-2 (B7-2₂₂₅) and pPIC9-B7-2₂₂₅-scFv(FRP5) (lower panel) encoding the B7-2 fragment fused to the scFv(FRP5) single chain antibody domain specific for ErbB2. Expression in the yeast Pichia pastoris is regulated by the alcohol oxidase promoter (AOX1) and is directed to the extracellular space via the yeast α-factor secretion signal (α). M, c-Myc tag; H, polyhistidine tag. (B) SDS-PAGE analysis of B7-2₂₂₅-scFv(FRP5) fusion protein. Lane 1, Coomassie-stained B7-2₂₂₅-scFv(FRP5) protein purified from Pichia pastoris culture supernatants; lanes 2 and 3, immunoblot analysis of purified B7-2₂₂₅-scFv(FRP5) (lane 2) and B7-2₂₂₅-scFv(FRP5) after treatment with protein N-gycosidase F (lane 3) with monoclonal antibody 9E10 specific for the C-terminal c-Myc tag of the fusion protein. M, molecular weight standards (kDa).

FIG. 2 Binding of B7-2₂₂₅-scFv(FRP5) to CTLA-4. The binding of B7-2₂₂₅-scFv(FRP5) to CHO cells (A) and CHO-CTLA-4 cells stably transfected with a human CTLA-4 cDNA (A, B, C) in the absence (A, B) or presence of a 50-fold molar excess of soluble CTLA-4 protein (C) was detected by FACS analysis with monoclonal antibody 9E10 and FITC-labeled (A) or PE-labeled (B, C) goat anti-mouse IgG.

FIG. 3 Binding of B7-2₂₂₅-scFv(FRP5) to ErbB2. (A) Immunoblot analysis with monoclonal antibody 9E10 of B7-2₂₂₅-scFv(FRP5) protein precipitated with glutathione-coupled agarose beads after incubation with a bacterially expressed glutathione S-transferase (GST)—ErbB2 fusion protein (lane 2) or a GST control protein (lane 3). M, molecular weight standards (kDa). (B) The binding of B7-2₂₂₅-scFv(FRP5) to murine HC11 cells and HC11-ErbB2 cells stably transfected with a human ErbB2 cDNA was detected by FACS analysis with monoclonal antibody 9E10 and FITC-labeled goat anti-mouse IgG.

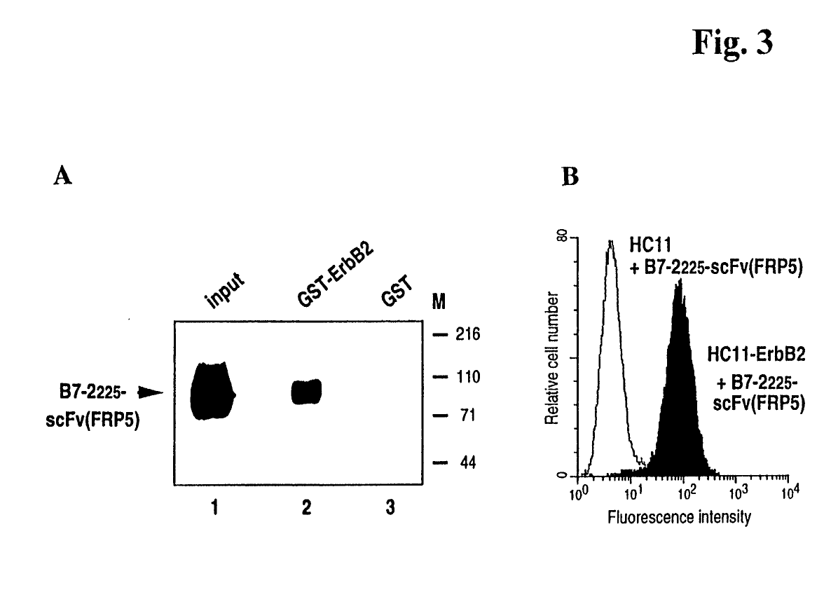

FIG. 4 Costimulation of syngeneic T cells by B7-2₂₂₅-scFv(FRP5). (A) HC11-ErbB2 cells and primary T cells from Balb/c mice pre-stimulated with PMA and IL-2 were incubated in the presence or absence of 10 ng/ml of purified B7-2₂₂₅-scFv(FRP5) as indicated. Proliferation of cells was measured by [³H]-thymidine incorporation. (B) Cells were treated as described in (A) with or without the addition of 2.5 μg/ml soluble CTLA-4 protein or 0.5 μg/ml anti-B7-2 antibody as indicated. Each value was determined in triplicates. The standard deviation is represented by error bars.

FIG. 5 Costimulation by B7-2₂₂₅-scFv(FRP5) is dependent on the binding to cell-surface ErbB2. (A) HC11-ErbB2 cells were incubated with pre-stimulated T cells as described in the legend of FIG. 4 in the presence of increasing concentrations of purified B7-2₂₂₅-scFv(FRP5) or B7-2₂₂₅ as indicated. Control cells were treated in the absence of recombinant proteins. Proliferation of cells was measured by [³H]-thymidine incorporation. (B) HC11-ErbB2 cells or parental HC11 cells were treated with 1 μg/ml B7-2₂₂₅-scFv(FRP5), incubated with a 5-fold excess of pre-stimulated syngeneic T cells as indicated and proliferation was measured by [³H]-thymidine incorporation. Controls show the background of [³H]-thymidine incorporation in the absence of T cells or target cells. Each value was determined in triplicates. The standard deviation is represented by error bars.

### EXAMPLE 1

Construction of the B7-2₂₂₅-scFv(FRP5) Fusion Gene

A fusion gene encoding the extracellular domain of human B7-2 (amino acids 1 to 225, referred to as B7-2₂₂₅), the ErbB2 specific scFv(FRP5), and synthetic sequence tags facilitating immunological detection and affinity purification was inserted into the AvrII and NotI restriction sites of the yeast expression vector pPIC9 (Invitrogen). In this vector expression of the gene is controlled by the methanol-inducible alcohol oxidase 1 (AOX1) promoter and the gene product is secreted into the medium via an N-terminal α-factor secretion signal. pPIC9 also contains a functional histidinol dehydrogenase (HIS4) gene for positive selection in the Pichia pastoris HIS4 mutant strain GS115 (Invitrogen). The B7-2₂₂₅ cDNA was derived from total RNA of human peripheral blood mononuclear cells (PBMC) by reverse transcription followed by PCR using the oligonucleotides B7-2-sense 5′-AAAAGTCGACGCTAGCGCTGCTCCTCTG-3′ (SEQ ID NO:1) and B7-2-antisense 5′-AAAACTCTAGAGATCTATCGATAGGAATGTGGTCTGG-3′ (SEQ ID NO:2), which introduce Sall and NheI restriction sites at the 5′-end and Clal, BgIII, and Xbal restriction sites at the 3′-end of the PCR product. The B7-2₂₂₅ cDNA fragment, the cDNA encoding the scFv(FRP5) (Wels et al., Bio/Technology, 1992), and a synthetic sequence encoding the Myc tag recognized by the monoclonal antibody (Mab) 9E10 (Evan, G.I., et al., 1985) as well as a polyhistidine tag were assembled into a single open reading frame and subsequently inserted into the expression vector pPIC9. As a control a similar B7-2₂₂₅ gene lacking the scFv(FRP5) domain was constructed. The integrity of the constructs was confirmed by restriction analysis and DNA sequencing.

### EXAMPLE 2

a) Cell Lines and Cell Culture Conditions

CHO cells and CHO-CTLA-4 cells stably transfected with a human CTLA-4 cDNA were maintained in MEMα with deoxyribonucleosides (Gibco BRL), containing 2 mM glutamine, 50 μM β-mercaptoethanol, 10% heat-inactivated fetal bovine serum (FBS), and 1 mg/ml G418 (CHO-CTLA-4). Balb/c derived HC11 mouse mammary epithelial cells and HC11-ErbB2-cells (HC11 R1 #11) stably transfected with a human erbB2 cDNA were grown in RPMI 1640 supplemented with 8% FBS and 5 μg/ml bovine insulin as described (Hynes, N. E., et al., 1990). The Pichia pastoris GS 115 yeast cells (Invitrogen) were propagated in buffered minimal glycerol-complex medium (BMGY) and expression of recombinant proteins was induced in buffered minimal methanol-complex medium (BMMY) according to the distributor's recommendation.

b) Expression and Purification of Recombinant Proteins

Construction and Expression in Yeast of the Chimeric Fusion Protein B7-2₂₂₅-scFv(FRP5)

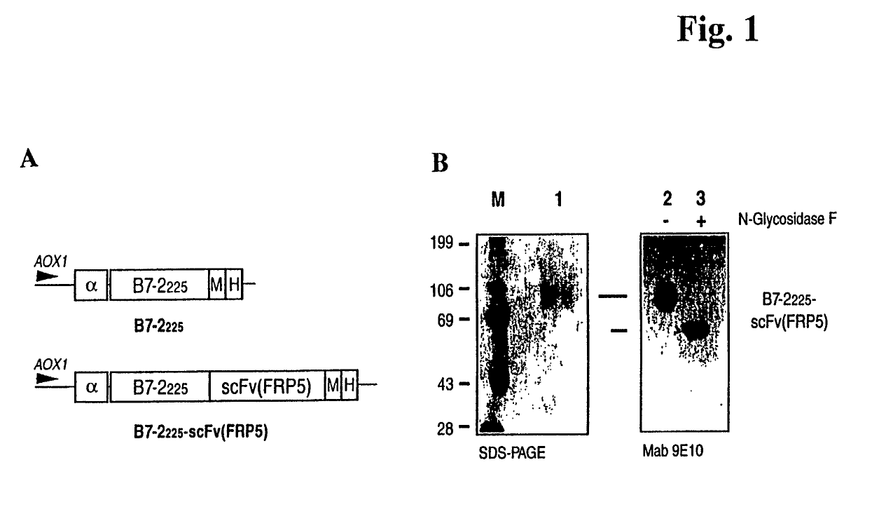

A chimeric gene encoding the extracellular domain of human B7-2 (amino acids 1 to 225, referred to as B7-2₂₂₅) fused to the ErbB2 specific scFv(FRP5) antibody domain (Wels et al., Bio/Technology, 1992) was constructed and inserted into the yeast expression vector pPIC9 shown in FIG. 1A. The resulting plasmid pPIC9-B7-2₂₂₅-scFv(FRP5) encodes under the control of the methanol inducible alcohol oxidase 1 (AOX1) promoter a chimeric fusion protein termed B7-2₂₂₅-scFv(FRP5), which consists of an N-terminal a-factor secretion signal from yeast, amino acids 1 to 225 of human B7-2, the scFv(FRP5) antibody domain, the Myc epitope recognized by Mab 9E10 (Evan et al., 1985), and a polyhistidine cluster facilitating the purification of the recombinant protein via Ni²⁺ affinity chromatography.

The B7-2₂₂₅-scFv(FRP5) protein was expressed in the Pichia pastoris strain GS115, and purified via Ni²⁺-affinity chromatography and gel filtration. The yield of soluble recombinant protein purified from 11 of yeast culture supernatant was typically 0.5 mg. SDS-PAGE and Mab 9E10 immunoblot analysis of the purified material revealed a purity of greater than 90% after two purification steps (FIG. 1B). The B7-2₂₂₅-scFv(FRP5) molecule is present as a monomer in yeast culture supernatants and in purified fractions as determined by SDS-PAGE under non-reducing conditions (data not shown). In contrast to the calculated molecular weight of 55.95 kDa the protein migrates as a smear of bands with apparent molecular masses of approximately 80 to 110 kDa in SDS-PAGE under reducing conditions (FIG. 1B). N-glycosidase F treatment of the protein reduced the apparent molecular mass to approximately 60 kDa indicating that the higher apparent molecular mass of yeast expressed B7-2₂₂₅-scFv(FRP5) protein is mainly due to post-translational modification by N-linked glycosylation.

Linearized pPIC9-B7-2₂₂₅ and pPIC9-B7-2₂₂₅-scFv(FRP5) plasmid DNAs were used for the transformation of Pichia pastoris GS115 cells by electroporation (Scorer, C. A., et al., 1994). His4₊/methanol-utilization⁺ (mut₊) yeast colonies were isolated on selection media following established protocols (Barr, K. A., et al., 1992) and upon induction with methanol B7-2₂₂₅ or B7-2₂₂₅-scFv(FRP5) expressing clones were identified by immunoblot analysis of culture supernatants using Mab 9E10. For expression at a larger scale a single colony each was grown to an OD₆₀₀ of 3 in BMGY medium, pH 8, the medium was exchanged with methanol-containing BMMY medium, pH 8, and protein expression was induced for 72 h at 30° C. Yeast cells were removed by centrifugation at 20,000 g. Supernatants containing the soluble B7-2₂₂₅ and B7-2₂₂₅-scFv(FRP5) fusion proteins were passed through a 45 μm filter, applied onto a Ni²⁺-saturated chelating sepharose column (Pharmacia Biotech) and the recombinant proteins specifically bound via their C-terminal polyhistidine tag were eluted with 250 mM imidazole in PBS. The B7-2₂₂₅-scFv(FRP5) protein was purified further by gel filtration using a Superdex 200 column (Pharmacia Biotech), fractions containing the fusion protein were identified by SDS-PAGE and immunoblotting with Mab 9E10, pooled, concentrated by ultrafiltration, and dialyzed against PBS. Post-translational modification of B7-2₂₂₅-scFv(FRP5) protein from yeast was analyzed in a dcglycosylation reaction. 0.2 μg of purified fusion protein were heated to 100° C. for 10 min in PBS containing 0.1% SDS. Triton X-100 was added to a final concentration of 1% and the protein was incubated with 1 U of N-glycosidase F (Boehringer Mannheim GmbH, DE) for 16 h at 37° C. in a total reaction volume of 100 μl. The sample was then analyzed by SDS-PAGE and immunoblotting with Mab 9E10.

### EXAMPLE 3

Binding Assays

a) Purified B7-2₂₂₅-scFv(FRP5) Specifically Binds to CTLA-4 Expressing Cells

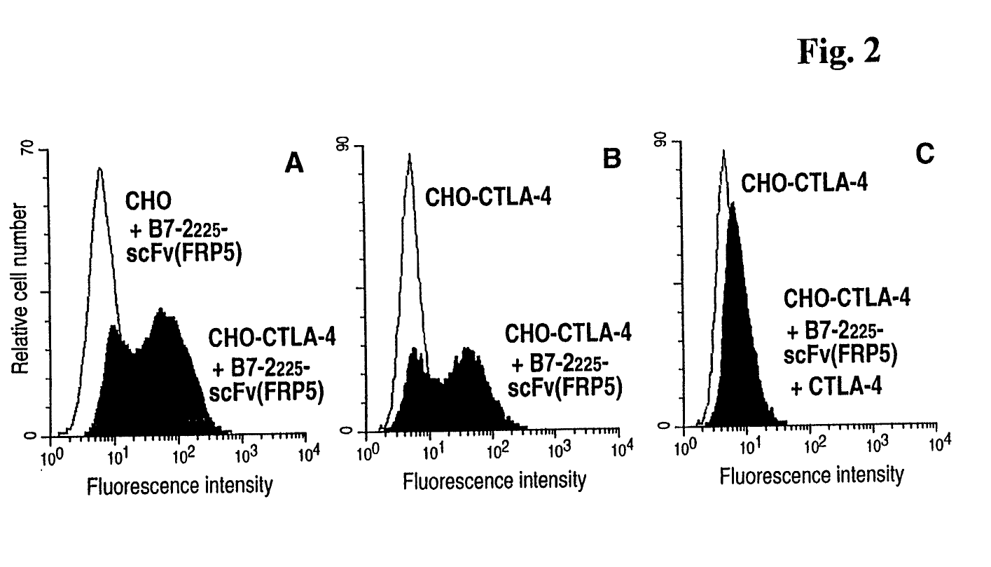

B7-2 binds to the B7 counter-receptors CD28 and CTLA-4 on the surface of T cells (Hathcock et al., 1993; Freeman et al., Science, 1993; Azuma et al., 1993). To investigate the functionality of the B7-2 domain the binding of the recombinant B7-2₂₂₅-scFv(FRP5) to CTLA-4 on the surface of cells was tested by FACS analysis. CHO-CTLA-4 cells stably transfected with a human CTLA-4 CDNA were incubated with B7-2₂₂₅-scFv(FRP5) and specifically bound fusion protein was detected with Mab 9E10 and FITC-labeled goat anti-mouse IgG. The results are shown in FIG. 2A. Significant binding of B7-2₂₂₅-scFv(FRP5) to CHO-CTLA-4 cells but not to CHO control cells could be detected. Comparable results were obtained with the B7-2₂₂₅ protein lacking the scFv domain and an anti-CTLA-4 antibody. The specificity of the B7-2₂₂₅-scFv(FRP5) binding to CTLA-4 was further confirmed in a competition assay. Similar to the soluble B7-2 protein a recombinant protein comprising amino acids 1 to 125 of human CTLA-4 was expressed in Pichia pastoris and purified from culture supernatants. CHO-CTLA-4 cells were incubated with B7-2₂₂₅-sceFv(FRP5) in the presence or absence of a 50-fold molar excess of soluble CTLA-4 protein as a specific competitor and the binding of B7-2₂₂₅-scFv(FRP5) was investigated by FACS analysis with Mab 9E10 and PE-labeled goat anti-mouse IgG. As shown in FIG. 2B and C soluble CTLA-4 protein almost completely blocked the binding of B7-2₂₂₅-scFv(FRP5) to CHO-CTLA-4 cells. These data indicate that the B7-2 domain of B7-2₂₂₅-scFv(FRP5) is functionally active and interacts specifically with a B7 counter-receptor.

The binding of B7-2₂₂₅-scFv(FRP5) to the B7 counter-receptor CTLA-4 was determined by FACS analysis using CHO-CTLA-4 cells and parental CHO cells as a control. 5×10⁵ trypsinized cells were incubated for 45 min at 4° C. with 0.1 or 1 μg of B7-2₂₂₅-scFv(FRP5) protein, followed by incubation with 3 μg of Mab 9E10 and FITC- or PE-labeled goat anti-mouse IgG (PharMingen) for 30 min. Binding of B7-2₂₂₅-scFv(FRP5) was detected using a FACScan (Becton-Dickinson). Similarly, the binding of B7-2₂₂₅-scFv(FRP5) to ErbB2 expressing HC11-ErbB2 mouse mammary epithelial cells was determined by FACS analysis. Binding of B7-2₂₂₅-scFv(FRP5) to ErbB2 was also tested using a recombinant glutathione S-transferase (GST) fusion protein which contains an N-terminal portion of the ErbB2 protein and is recognized by the ErbB2 specific Mab FRP5. Bacterially expressed GST or GST-ErbB2 fusion proteins (10 μg) were bound to 200 μl each of glutathione-coupled agarose beads (Sigma). The beads were incubated with 4 μg B7-2₂₂₅-scFv(FRP5), washed with PBS, and specifically bound proteins were analyzed by SDS-PAGE and immunoblotting with a Mab binding specifically to the B7-2₂₂₅-scFv(FRP5) protein.

### EXAMPLE 4

Cell Proliferation Assays

a) B7-2₂₂₅-scFv(FRP5) Provides Costimulation for the Proliferation of Syngeneic T Cells

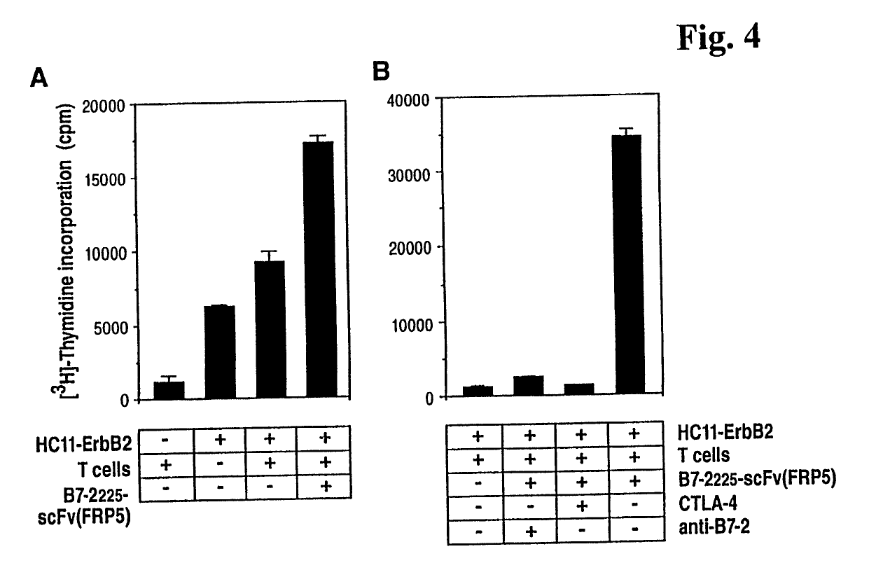

The chimeric B7-2₂₂₅-scFv(FRP5) protein is bispecific since it binds to CTLA-4 and to ErbB2 on the surface of cells. Several reports have demonstrated that human B7-1 or B7-2 can interact functionally with the murine B7 counter-receptors CD28 or CTLA-4, and vice versa (Freeman, G. J., et al., J. Exp. Med., 1993; Cai, Y. C., et al., 1995). In order to determine whether purified B7-2₂₂₅-scFv(FRP5) presented on the surface of cells can provide costimulation for T-cell proliferation, a syngeneic lymphocyte reaction (SLR) was performed using primary T cells from Balb/c mice pre-stimulated with PMA and IL-2, and murine HC11-ErbB2 cells which are of Balb/c origin and express human ErbB2 on their surface. HC11-ErbB2 cells were treated with B7-2₂₂₅-scFv(FRP5) (10 ng/ml) and subsequently incubated with a 5-fold excess of pre-stimulated T cells. B7-2₂₂₅-scFv(FRP5) treatment resulted in a strong increase in T-cell proliferation in comparison to cells treated in the absence of the fusion protein (FIG. 4A). The addition of a 500-fold molar excess of soluble CTLA-4 or a 30-fold molar excess of the inhibitory anti-B7-2 antibody FUN-1 (PharMingen) reduced completely the stimulatory effect of B7-2₂₂₅-scFv(FRP5) (FIG. 4B). This indicates that the observed stimulation of T-cell proliferation is due to specific interaction of the B7-2 domain with its cognate counter-receptor on the T cells.

b) Binding of B7-2₂₂₅-scFv(FRP5) to Cell-Surface ErbB2 is Required for Costimulation of T-cell Proliferation

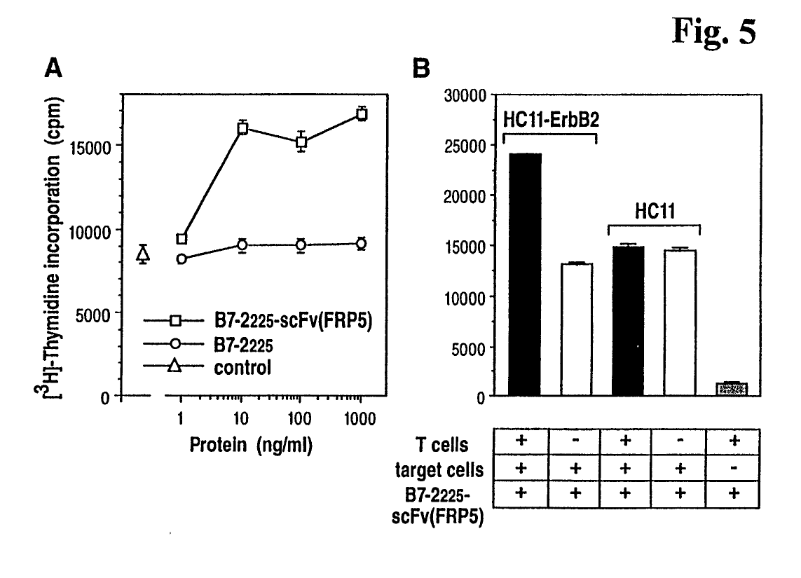

Cell-surface bound B7-2₂₂₅-scFv(FRP5) provides a costimulatory signal for T-cell proliferation. To investigate whether the presentation on the cell surface is necessary for the costimulatory activity of B7-2₂₂₅-scFv(FRP5) a SLR experiment with HC11-ErbB2 cells and syngeneic T cells was performed as described above either in the presence of increasing concentrations (1 to 1000 ng/ml) of purified B7-2₂₂₅-scFv(FRP5) or a similar B7-2₂₂₅ protein lacking the ErbB2 specific antibody domain. As shown in FIG. 5A, B7-2₂₂₅ which was present in the incubation but is unable to bind to the surface of HC11-ErbB2 cells did not stimulate T-cell proliferation, whereas the B7-2₂₂₅-scFv(FRP5) molecule at concentrations of 10 ng/ml or higher strongly enhanced T-cell proliferation. The dependency of the costimulatory activity of B7-2₂₂₅-scFv(FRP5) on the binding to cell-surface ErbB2 was confirmed in a similar SLR experiment with HC11-ErbB2 and parental HC11 cells. HC11-ErbB2 but not HC11 cells in the presence of B7-2₂₂₅-scFv(FRP5) (1 μg/ml) led to a strong stimulation of T-cell proliferation indicating that presentation of the chimeric protein on the cell surface of ErbB2 expressing cells is required for its costimulatory activity (FIG. 5B). These data show that the B7-2₂₂₅-scFv(FRP5) molecule is highly specific for ErbB2 expressing target cells and does not enhance T-cell proliferation in a reaction with ErbB2-negative cells when it is present only in soluble form.

Spleen cells from Balb/c mice were depleted of red blood cells by hypotonic lysis with NH₄Cl and subsequently passed through a nylon-wool syringe as described (Coligan, J. E., et al., 1993). The enriched cell preparation contained more than 85% T cells (TCR⁺), less than 5% B cells (Ig⁺), and about 10% other cells as determined by FACS analysis. Enriched primary T cells were pre-stimulated for 48 h in medium containing 10 ng/ml PMA (Sigma) and 50 IU/ml recombinant human IL-2 (Boehringer Mannheim GmbH, DE). 2×10⁴ cells/well of mitomycin-treated HC11 or HC11-ErbB2 cells were incubated for 2 h with 1, 10, 100, or 1000 ng/ml of the B7-2₂₂₅-scFv(FRP5) fusion protein in 96 well plates. Control cells were treated with the B7-2₂₂₅ protein lacking the scFv(FRP5) domain or left untreated. 1×10⁵ pre-stimulated T cells were added to each well and the cells were incubated further for 2 h in a total volume of 200 μl/well of RPMI medium supplemented with 8% FBS and 20 IU/ml recombinant human IL-2. The cells were pulsed with 0.25 μCi/well [³H]-thymidine (Du Pont) for 20 h, and the incorporation of [³H]-thymidine was measured with a liquid scintillation counter (Beckman).

### LIST OF REFERENCES

Alvarez-Vallina, L., et al., Eur. J. Immunol. 26 (1996) 2304-2309

Azuma, M., et al., Nature 366 (1993) 76

Barr, K. A., et al., Pharm. Eng. 12 (1992) 48

Baskar, S., et al., Proc. Natl. Acad. Sci. USA 90 (1993) 5687

Brunschwig, E. B., et al., J. Immunol. 155 (1995) 5498

Cai, Y. C., et al., Immunity 3 (1995) 417

Chen, L., et al., Cell 71 (1992) 1093

Coligan, J. E., et al., Curr. Prot. Immunol. 2 (1993) 3.2.1

Cromme, F. V., et al., J. Exp. Med. 179 (1994) 335

Doyle, A., et al., J. Exp. Med, 161 (1985) 1135

Evan, G. I., et al., Mol. Cell. Biol. 5 (1985) 3610

Freeman, G. J., et al., J. Exp. Med. 178 (1993) 2185

Freeman, G. J., et al., Science 262 (1993) 909

Gajewski, T. F., et al., J. Immunol. 156 (1996) 2909

Galvin, F., et al., J. Immunol. 149 (1992) 3802

Gimmi, C. D., et al., Proc. Natl. Acad. Sci. USA 88 (1991) 6575

Gimmi, C. D., et al., Proc. Natl. Acad. Sci. USA 90 (1993) 6586

Greenberg, P. D., Adv. Immunol. 49 (1991) 281

Harding, F. A., et al., Nature 356 (1992) 607

Hathcock, K. S., et al., Science 262 (1993) 905

Hodge, J. W., et al., Cancer Res. 54 (1994) 5552

Hynes, N. E., et al., Mol. Cell. Biol. 10 (1990) 4027

Hynes, N. E., Semin. Cancer Biol. 4 (1993) 19

Lassam, N., and Jay, G., J. Immunol. 143 (1989) 3792

Ledbetter, J. A., et al., Blood 75 (1990) 1531

Li, Y., et al., J. Immunol. 153 (1994) 421

Linsley, P. S., et al., J. Biol. Chem. 270 (1995) 15417

Linsley, P. S., et al., J. Exp. Med. 173 (1991) 721

Linsley, P. S. et al., Immunity 1 (1994) 793

Lundberg, A., et al., Blood 82 (1993) 123a

Matulonis, U., et al., J. Immunol. 156 (1996) 1126

McHugh, R. S., et al., Proc. Natl. Acad. Sci. USA 92 (1995) 8059

Melief, C. J., Adv. Cancer Res. 58 (1992) 143

Moritz, D., et al.. Proc. Natl. Acad. Sci. USA 91 (1994) 4318

Peles, E., and Yarden, Y., Bioessays 15 (1993) 815

Restifo, N. P., et al., J. Exp. Med. 177 (1993) 265

Rudd, C. E., et al., Immunol. Today 15 (1994) 225

Scorer, C. A., et al., Bio/Technology 12 (1994) 181

Tan, P. C., et al., J. Exp. Med. 177 (1993) 165

Townsend, S. E., and Allison, J. P., Science 259 (1993) 368

Wels, W., et al., Bio/Technology 10 (1992) 1128

Wels, W., et al., Cancer Res. 52 (1992) 6310

Yang, G., et al., J. Immunol. 154 (1995) 2794

### Sequence Listing

(1) General Information

(i) Applicant:

(A) Name: Boeheringer Mannheim GMBH

(B) Street: Sandhofer Str. 116

(C) City: Mannheim

(E) Country: Germany

(F) Postal Code (Zip): D-68305

(G) Telephone: 08856/60-3446

(H) Telefax: 08856/60-3451

(ii) Title of Invention: Costimuation of T-cell proliferation by a chimeric bispecific costimulatory protein

(iii) Number of Sequences: 2

(iv) Computer Readable Form:

(A) Medium Type: Floppy disk

(B) Computer: IBM PC compatible

(C) Operating System: PD-DOS/MS-DOS

(D) Software: PatentIn Release #1.0, Version #1.3B (EPO)

(2) Information for Seq. ID No: 1:

(i) Sequence Characteristics:

(A) Length: 28 base pairs

(B) Type: nucleic acid

(C) Strandedness: single

(D) Topolgy: linear

(ii) Molecule Type: other nucleic acid

(A) Description: /desc=“primer”

(xi) Sequence Description: Seq ID No: 2: AAAAGTCGAC GCTAGCGCTG CTCCTCTG

(2) Information for Seq ID No: 2:

(i) Sequence Characteristics:

(A) Length: 37 base pairs

(B) Type: nucleic acid

(C) Strandedness: single

(D) Topolgy: linear

1. A method for the manufacture of a therapeutic agent consisting of a bispecific fusion protein in solution, which consists of
a) a binding domain which recognizes a specific surface molecule on a target cell, covalently linked to
b) a domain capable of costimulation of T cell proliferation,
for a specific costimulation of a T cell directed against said target cell of a patient.

2. The method according to claim 1, wherein said binding domain binds to a growth factor or a cytokine receptor expressed on the surface of said target cell.

3. The method according to claim 2, wherein said binding domain is a binding domain specific for an ErbB receptor tyrosine kinase expressed on the surface of said target cell.

4. The method according to claim 1, wherein said binding domain binds to an antigen of a pathogen (e.g., virus, bacterium, yeast, fungi) expressed on the surface of said target cell.

5. The method according to claims 1 to 4, wherein said costimulatory domain is a binding domain of a B7 molecule, binding its counter-receptor on the surface of a T cell.

6. The method according to claims 1 to 4, wherein said costimulatory domain is a binding domain of CD40L binding its counter-receptor on the surface of T cells.

7. The method according to claims 1 to 6, wherein said target cell is a tumor cell.

8. The method according to claims 1 to 7, wherein said target cell is a cell infected by a pathogen (e.g., virus, bacterium, yeast, fungi).

9. The method according to claims 1 to 8, wherein said T cell is a syngeneic T cell.

10. The method according to claims 1 to 9, wherein said T cell is a patient-derived tumor infiltrating lymphocyte, a lymphokine-activated killer cell or a cytotoxic T cell.

11. The method according to claims 1 to 10, wherein said T cell is a cytotoxic T cell and said patient is treated with IL-2 and subsequently treated with said bispecific fusion protein.

12. Use of a soluble bispecific fusion protein consisting of
a) a binding domain which recognizes a specific surface molecule on a target cell, covalently linked to
b) a domain capable of costimulation of T cell proliferation,
for a specific costimulation of a T cell directed against said target cell.
